(19) United States
(12) Patent Application Publication
(10) Pub. No.: US 2013/0171710 A1
(43) Pub. Date: Jul. 4, 2013

(54) Anaerobic Reactor

(71) Applicant: Andrew Prebble, Bucharest (RO)

(72) Inventor: Andrew Prebble, Bucharest (RO)

(73) Assignee: ADVANCED BIOGAS TECHNOLOGIES SRL, Bucharest (RO)

(21) Appl. No.: 13/516,056

(22) PCT Filed: Dec. 14, 2010
(86) PCT No.: PCT/GB2010/002271

§ 371 Date: Nov. 26, 2012

(30) Foreign Application Priority Data
Dec. 14, 2009 (GB) 0921836.3

(51) Int. Cl. C12P 5/02

(52) U.S. Cl. CPC C12P 5/023
USPC 435/167; 435/289.1; 435/287.1

(57) ABSTRACT

An anaerobic reactor comprising two or more discrete reaction chambers arranged one above the other is disclosed. The reactor may be in the form of a tank separated into discrete chambers by means of solid members, or the reactor may be in the form of separate and stackable chambers. Additionally, the reactor may be a packed bed reactor, a fluidised bed reactor, or a hybrid reactor comprising at least one packed bed reaction chamber and at least one fluidised bed reaction chamber. Use of an anaerobic reactor according to the invention enables an increase in the yield of biogas that can be produced per unit area of land occupied by an anaerobic reactor. A method of producing biogas is also disclosed, the method comprising providing an anaerobic reactor according to the invention, providing input biomass, carrying out anaerobic digestion of the biomass in the reactor, and collecting the biogas produced.

The present invention relates to a reactor for the anaerobic production of biogas, in particular an anaerobic reactor comprising more than one packed or fluidised bed, or layer.

Anaerobic digestion is a series of processes in which microorganisms break down biodegradable material in the absence of oxygen. There are three principal products of anaerobic digestion: biogas, digestate and water. Biogas, produced by anaerobic digestion, or fermentation of biodegradable materials, is comprised primarily of methane and carbon dioxide. The methane in biogas can be burned to produce heat and electricity. Digestate contains the solid remnants of the original input material to the digesters that the microbes cannot use. It also comprises the mineralised remains of the dead bacteria from within the digesters. The water produced by anaerobic digestion systems originates from the moisture content of the input biomass, as well as water produced during the digestion process.

Almost any organic material can be processed with anaerobic digestion, including biodegradable waste materials such as waste paper, grass clippings, leftover food, sewage, animal waste and liquid waste. Anaerobic digesters can also be fed with specially grown energy crops such as silage for dedicated biogas production. Anaerobic digestion is particularly suited to wet organic material and is commonly used for effluent and sewage treatment. Anaerobic digestion is widely used to treat wastewater sludges and organic waste because it results in volume and mass reduction of the input material—the process that can greatly reduce the amount of organic matter which might otherwise be destined to be landfilled or burnt in an incinerator. In addition, anaerobic digestion is used in the production of renewable energy, because the process produces a methane and carbon dioxide-rich biogas suitable for energy production. Methane and power produced in anaerobic digestion facilities can be utilized to replace energy derived from fossil fuels, and hence reduce emissions of greenhouse gases. This is due to the fact that the carbon in biodegradable material is part of a carbon cycle. The carbon released into the atmosphere from the combustion of biogas has been removed from the atmosphere by plants, in order for them to grow, in the recent past. This can have occurred within the last decade, but more typically within the last growing season. If the plants are re-grown, taking the carbon out of the atmosphere once more, the system will be carbon neutral. This contrasts to carbon in fossil fuels that has been sequestered in the earth for many millions of years, the combustion of which increases the overall levels of carbon dioxide in the atmosphere. The nutrient-rich solids (digestate) remaining after anaerobic digestion are valuable as a fertiliser.

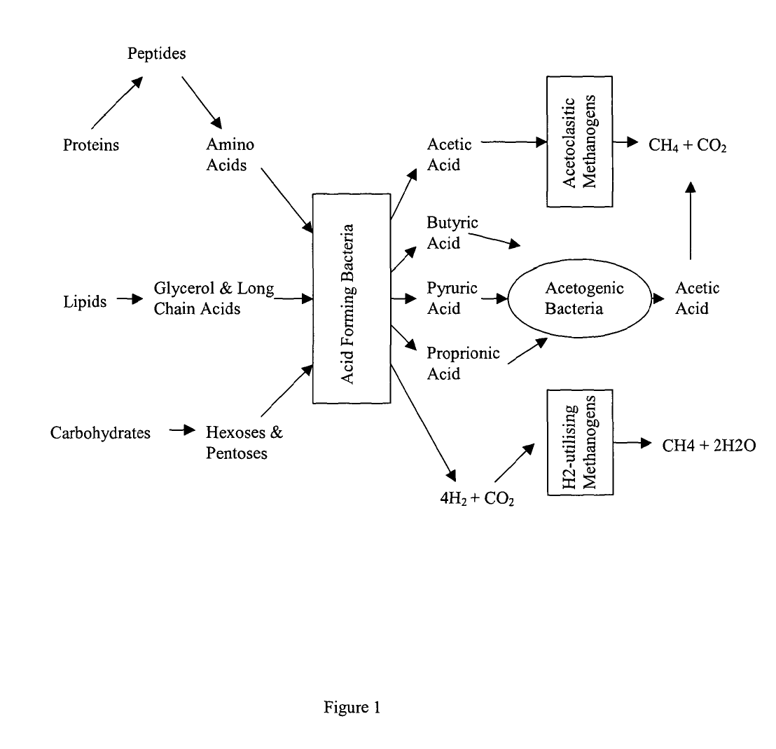

A number of different bacteria are involved in the process of anaerobic digestion. These include hydrolytic bacteria, organic acid-forming bacteria (acidogens); acetic acid-forming bacteria (acetogens); and methane-forming archaea (methanogens). These organisms feed upon the initial feedstock, which undergoes a number of different processes converting it to intermediate molecules including sugars, hydrogen and acetic acid before finally being converted to biogas (FIG. 1).

In general, the input material, or biomass, is made up of large organic polymers. Many bacteria are unable to utilise these large organic polymers, so the energy potential of the material is largely inaccessible. The anaerobic digestion process begins with bacterial hydrolysis of these larger organic molecules, such as proteins, lipids and carbohydrates, which are broken down to smaller molecules such as amino acids, glycerol, long-chain fatty acids and sugars. Bacterial hydrolysis therefore makes the input material available to other bacteria. Acidogenic bacteria then convert the products of hydrolysis (sugars, amino acids, and so on) into organic acids, such as volatile fatty acids. Hydrogen and carbon dioxide are also formed at this stage. Subsequently, acetogenic bacteria convert the resulting organic acids into acetic acid, along with additional ammonia, hydrogen and carbon dioxide. Finally, acetoclastic methanogens, convert the acetic acid to methane and carbon dioxide. Simultaneously, another class of methanogens (hydrogen-utilising methanogens) recombine the carbon dioxide and hydrogen into methane and water (see FIG. 1).

As indicated above, there are four classes of bacteria that make up anaerobic biomass. In order to carry out the anaerobic digestion process, it is necessary to retain the anaerobic biomass, comprising the four types of bacteria, within the reactor. Retaining the anaerobic bacteria within the reactor enables the reactor to operate at higher organic matter loading rates. A high rate anaerobic reactor has hydraulic retention times for the feed, or organic, material measured in hours, rather than days.

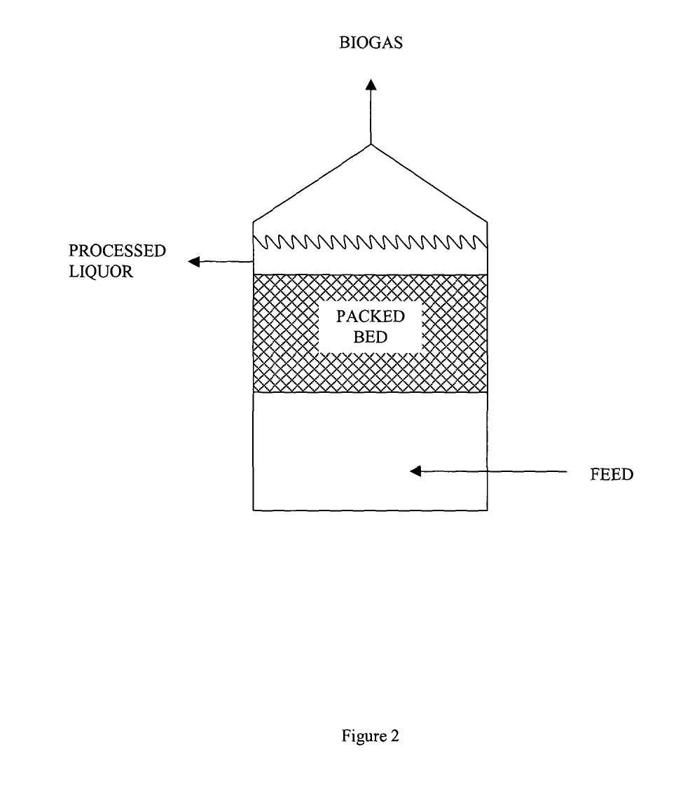

Containing the anaerobic bacteria within the anaerobic reactor has long been a problem. A number of reactor designs have been developed to overcome this problem. In particular, packed beds are used to retain the anaerobic bacteria within the reactor when digesting wastes with a relatively low solids content typically less than 1000 mg/l or lower (FIG. 2). Packed bed reactors employ a layer, or bed, of packing material, which may be randomly oriented or regular. Typically, a packed bed reactor comprises a bed of packing media, wherein input biomass, or feedstock, is fed in at the base of the packed bed and flows up through the packed bed, with the products of digestion (processed liquor and biogas) being removed from the top of the bed.

The principle purpose of the packing is to retain the anaerobic bacteria, particularly the methanogens since of the four classes of bacteria in the anaerobic bacteria the metanogens are the slowest growing. Methanogens prefer to associate with a surface, and therefore a packing media with a high surface area to volume ratio provides an ideal environment. Packed beds allow the retention of anaerobic bacteria within the reactor and therefore avoid delays spent waiting for bacteria to grow, so enabling the reactor to operate at higher loading rates and shorter hydraulic retention times. However, packed bed reactors have some disadvantages.

The key limitation of the packed bed design is that the combined up-flow velocity (that is, the simple arithmetic combination of the velocity of the flow of liquid and of the gas generated by the bacteria) through the packed bed needs to be kept low enough to avoid stripping the methanogens from the packing media. Ideally, the combined up-flow velocity is kept below about 80 to 90 m/day. In a fully loaded reactor, this limits the depth of the packed bed to no more than 2 metres to 3 metres, depending on the feed strength and volume of the organic matter fed into the reactor. This limitation on bed depth strictly limits the amount of input material, or organic matter, that may be processed, and therefore limits the volume of biogas that may be produced from a given reactor footprint. This limitation on the potential for biogas production per unit area of reactor footprint therefore strictly limits the potential to reduce the capital cost of anaerobic reactors per unit of gas produced.

Attempts have been made to increase the yield of biogas from a given reactor footprint by arranging gas collectors vertically through a reactor to reduce the volume of gas emanating from the top of the reactor. However, this does not provide a satisfactory solution to the problem, because high concentrations of the volatile acids produced by the acidogenic bacteria in the biomass create a toxic environment for other anaerobic bacteria by reducing the pH. Therefore, the load that can be applied to the lower portions of the reactor is restricted, and as a result the volume of biogas that may be produced from a given reactor footprint (that is, the area of land occupied by the reactor) remains limited.

We have recognised, therefore, that there is a need for an anaerobic reactor which can produce higher yields of biogas from a given reactor footprint.

According to the present invention in its broadest aspect, there is provided an anaerobic reactor comprising two or more discrete reaction chambers arranged one above the other.

The reaction chambers are separated from each other, except for the limited inter-connection necessary for pressure equalisation between the chambers. Pressure equalisation devices may be used to allow small quantities of processed liquor to pass between the chambers to equalise the pressure at the top of one chamber and the bottom of the chamber immediately above it. The chambers are not connected for the purposes of processing the liquor, and therefore, the reaction chambers can be considered to be discrete chambers. By “discrete chambers”, it is intended that the chambers are not interconnected, other than to the extent necessary for pressure equalisation. In particular, there is no flow of bacteria, biomass or of the products of anaerobic digestion between chambers. Each chamber operates independently of the other chamber(s) to produce biogas.

The anaerobic reactor may comprise an outer housing within which the two or more discrete reaction chambers are arranged one above the other. In such embodiments, the chambers may be separated from one another by a solid member, for example a solid plate, with each solid member separating the process of one chamber from the process of an adjacent chamber. The solid member(s) need not be structural because the outer housing can provide structural support to the discrete reaction chambers. Therefore, the solid plate should have sufficient strength to support itself, to repel the flow of feed directed towards it for mixing purposes, and to cope with the small variations in pressure between adjacent chambers, but may not have sufficient strength to support the anaerobic reactor. Hence, in a particularly preferred embodiment, the structure of the reactor can be considered as a standard tank, preferably a vertical, cylindrical tank, separated into discrete chambers by means of one or more solid members. Use of a standard tank separated into discrete chambers helps to minimise costs.

As a result of the arrangement of the discrete chambers, the pressure in the lower chambers is equivalent to the total “head pressure” imposed by the chambers above. Therefore, it is generally preferred to have at least one pressure regulating, or pressure sustaining, device positioned at the discharge from each chamber, in order to control the transition of the liquid and gas to atmospheric pressure. The pressure regulating device(s) may be in the form of a valve.

In embodiments in which solid member(s) are used to separate the process of one chamber from the process of an adjacent chamber, having a pressure equalisation device between adjacent chambers helps to prevent the collapse of the solid member separating the adjacent chambers.

Alternatively, the chambers can be separated by each chamber being separate and stackable. In this arrangement, it is necessary that the base of the reactor is of a suitable strength to carry the load of the chambers plus the load of the liquid in the digester.

The chambers can be arranged one above another in any suitable configuration or arrangement. The configuration of reaction chambers is such as to reduce, preferably substantially reduce, more preferably minimise, the area of land occupied by the reactor. Therefore, the arrangement or configuration of reaction chambers gives a reduction in the footprint of the reactor. In a particularly preferred embodiment, the reaction chambers are arranged substantially vertically one above the other (i.e. the reaction chambers are arranged in a substantially vertical stack). Where the reaction chambers are arranged in a substantially vertical stack, the reactor may comprise one or more such stacks. However, any suitable arrangement which gives a reduction in the reactor footprint may be used.

In one embodiment, the reactor is preferably a packed bed reactor, therefore each of the reaction chambers preferably comprises a packed bed. The packing, or media, is retained in position in each chamber for example by means of upper and/or lower grids or other constraining means. The reactor can therefore be considered a multibed reactor. It is preferred that each chamber comprises a feed distribution system, a packed bed, and means for biogas and liquid discharge.

In another embodiment, the reactor is a fluidised bed reactor. In contrast to the packed bed system described above, in a fluidised bed reactor the packing or support media is allowed to move around the designated space within each chamber in a fluidised manner. Conventionally, fluidised bed reactors employ media which is denser than water, for example sand, glass beads, carbon (in varying forms such as felt blocks) and the like. The material is fluidised by the flow of the feed liquor, typically by the upflow velocity of the feed liquor. Sufficient upflow velocity is required to effectively keep the media in suspension.

As an alternative to conventional fluidised bed media, it has been found that media intended for aerobic waste water treatment installations can be used in the reactor of the present invention. Such media perform the duty of a bioflim carrier whereby the anaerobic bacteria settle on or attach themselves to the various surfaces of the matrix provided by the varying media designs. (In the context of the present invention, a “biofilm” is understood to be a layer of a bacterial culture.) The designed matrix, whether regular or random, provides a high surface area to volume ratio (typically 300-900 m²/m³ but up to 3,000 m²/m³ in exceptional cases). Of the total surface area a significant proportion (usually 70%) is designated as protected i.e. not subject to erosion of the biofilm when the media bump together.

When these media are used, the filling fraction of the fluidised bed is 67% or less depending on the parameters of the process and the physical characteristics of the material being processed. The media preferably occupies about 55 to 67% of the total space available, so that there is sufficient space for the media to circulate freely.

Suitable media may be made from material that has a neutral, or close to neutral, buoyancy when in an aqueous environment. This helps ensure the media make good contact with the input biomass. Commonly, such media are made from a soft plastic (such as recycled polyethylene) and may be in the shape of a piece of tube with internal separating walls and fins on the outside. Such a shape helps maximise surface area whilst allowing liquid biomass to flow freely through and around the media. For example, Veolia Mass Transfer supplies such media for aerobic installations, which media has a much higher surface area to volume ratio than conventional media for fluidised beds. The Veolia media provides a surface area to volume ratio of up to 800-1,400 m²/m³ total area and 500-1200 m²/m³ protected area. These media are commercially available under the tradenames Kaldness K1™, Kaldness K2™, BiofilmChip M™ and BiofilmChip P™. These materials are specifically designed for use in a fluidised manner.

An alternative form of media suitable for use in the present invention comprises a variety of plastic bodies in the form of curved plates and/or hyperbolic paraboloids with a porous surface. The interior of the pores provides a high surface area that is protected from the reaction environment to help prevent erosion of the biofilm. The density of the plastic bodies and the average pore size of the porous surface can be adjusted during manufacture to suit the end application.

Multi Umwelttechnologie AG supplies such media, including Mutag BioChip™, for aerobic, anaerobic and anoxic processes. Mutag BioChip™ provides a protected surface area of approximately 3000 m²/m³. The curved shape of the Mutag BioChip™ helps ensure the liquid biomass can flow freely around the media and ensure it moves continuosly within the liquid biomass when in use within a reactor. These materials are also specifically designed for use in a fluidised manner.

The invention therefore also provides the use of media intended for aerobic waste water treatment installations in a multi-layered or multi-bed fluidised bed anaerobic reactor as described herein. Any media that has been designed for, or is suitable for, use as the support media for aerobic bacteria in aerobic waste water treatment stations may be used in this aspect of the invention. The above-described media commercially supplied by Veolia Mass Transfer and Multi Umwelttechnologie AG are particularly suitable, but any suitable media may be used.

When fluidised beds rather than packed beds are used, the reactor can be operated as a multi-layered fluidised bed reactor. It is predicted that a multi-layered fluidised bed reactor could produce up to two, three or four times greater biogas gas yields than a packed reactor because of the larger surface area in the fluidised beds being able to accommodate a greater population of bacteria. This is a significant advantage of a fluidised bed reactor. A further advantage of a fluidised bed type reactor is that with fluidised rather than packed beds, each bed is less likely to block and therefore the reactor is able to accommodate a higher level of solids loading i.e. greater than 1,000 mg/l.

In another embodiment, the reactor of the present invention may be a hybrid reactor comprising at least one packed bed reaction chamber and at least one fluidised bed reaction chamber. In such a reactor each of the reaction chambers can comprise either a packed bed or a fluidised bed, a feed distribution system, and means for biogas and liquid discharge. The hybrid reactor may comprise means to assess input biomass prior to processing and to divert the input biomass to either a packed bed reaction chamber or a fluidised bed reaction chamber depending on the nature of the input biomass. For example, liquid with a suspended solids content of from 500 to 1,000 mg/l could be diverted to a packed bed reactor, whilst liquid with a suspended solids content of greater than 1,000 mg/l could be diverted to a fluidised bed reactor.

In a preferred aspect, there is provided an anaerobic reactor, comprising two or more discrete reaction chambers arranged one above the other, wherein each reaction chamber comprises a feed distribution system, a packed bed, and means for biogas and liquid discharge.

Preferably each chamber is separated from the adjacent chamber(s), for example by means of a solid member such as a solid plate. The reaction chambers can be arranged one above another in any suitable configuration, for example they can be arranged in a substantially vertical configuration, so as to form one or more substantially vertical stacks.

In a further aspect, the invention provides an anaerobic reactor, comprising one or more discrete reaction chambers arranged one above the other, wherein each reaction chamber comprises a feed distribution system, a fluidised bed, and means for biogas and liquid takeoff. Preferably, the chambers are separated from one another as described herein. The reaction chambers can be arranged one above another in any suitable configuration, for example they can be arranged in a substantially vertical configuration so as to form one or more substantially vertical stacks. The fluidised beds may comprise conventional fluidised bed media as described above. Alternatively, the fluidised beds may comprise media intended for use in aerobic fluidised bed installations (that is, intended for aerobic waste water treatment installations), such as the Veolia Mass Transfer media Kaldness K1™, Kaldness K2™, BiofilmChip M™, and BiofilmChip P™. Or Mutag BioChip™ as available from Multi Umwelttechnologie AG.

There is also provided the use of a reactor according to the invention, preferably in the production of biogas.

In another aspect there is provided a method of producing biogas comprising providing an anaerobic reactor according to the invention; providing input biomass; carrying out anaerobic digestion of the biomass in the reactor; and collecting the biogas produced. If desired, the digestate so-produced can also be collected and used, for example as a fertiliser or in any other suitable application, or in the case of the digestion of liquid wastes, aerobically polished for discharge.

In methods using a packed bed reactor, the input biomass preferably comprises a liquid with a suspended solids content of from 500 to 1,000 mg/l, whereas in methods using a fluidised bed reactor, the input biomass preferably comprises a liquid with a suspended solids content of greater than 1,000 mg/l. In methods using a hybrid reactor, the input biomass comprising a liquid with a suspended solids content of from 500 to 1,000 mg/l is processed by a packed bed reaction chamber and the input biomass comprising a liquid with a suspended solids content greater than 1,000 mg/l is processed by a fluidised bed reaction chamber.

The method of producing biogas described above enables a method of increasing the yield of biogas that can be produced per unit area of land occupied by an anaerobic reactor.

The arrangement of the reaction chambers one above another in the reactor of the invention increases the volume of material that may be processed and therefore the volume of biogas that may be produced from a set reactor footprint. The volume of biogas that may be produced is increased by the factor of the number of reaction chambers, or layers, that are employed. The capital cost per unit of biogas produced is similarly reduced, albeit by a lesser factor. The invention has particular relevance where space is at a premium or the cost of land is high.

FIG. 1 is a schematic illustration of the anaerobic digestion process.

FIG. 2 shows the general structure of a conventional upflow packed bed reactor.

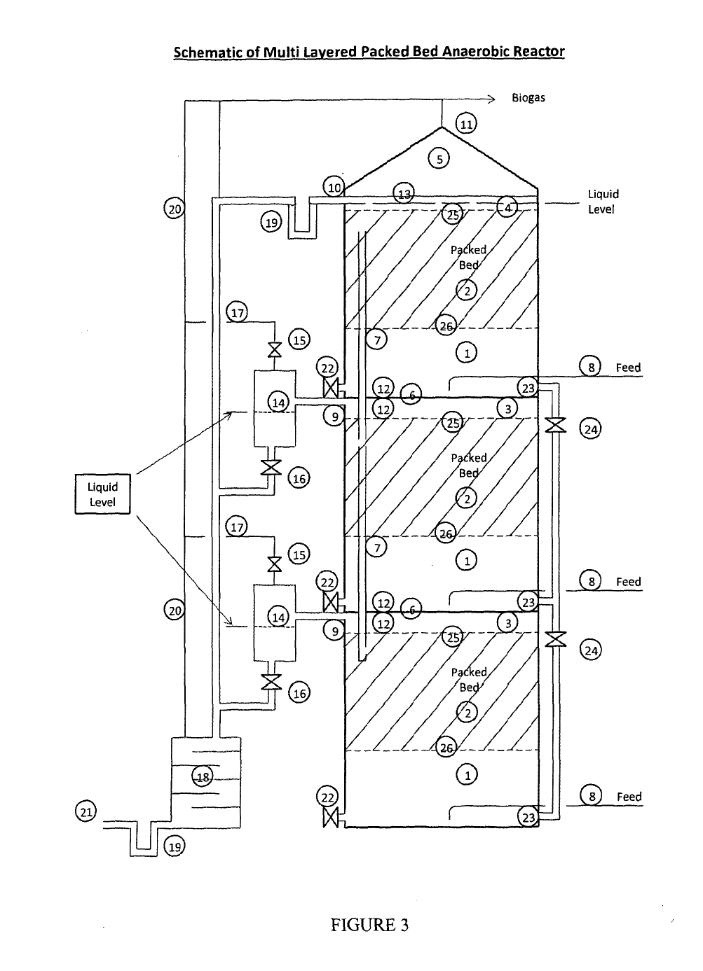

FIG. 3 shows the structure of a packed bed reactor according to the invention. The reactor is a multi-layered packed bed reactor, or multibed reactor.

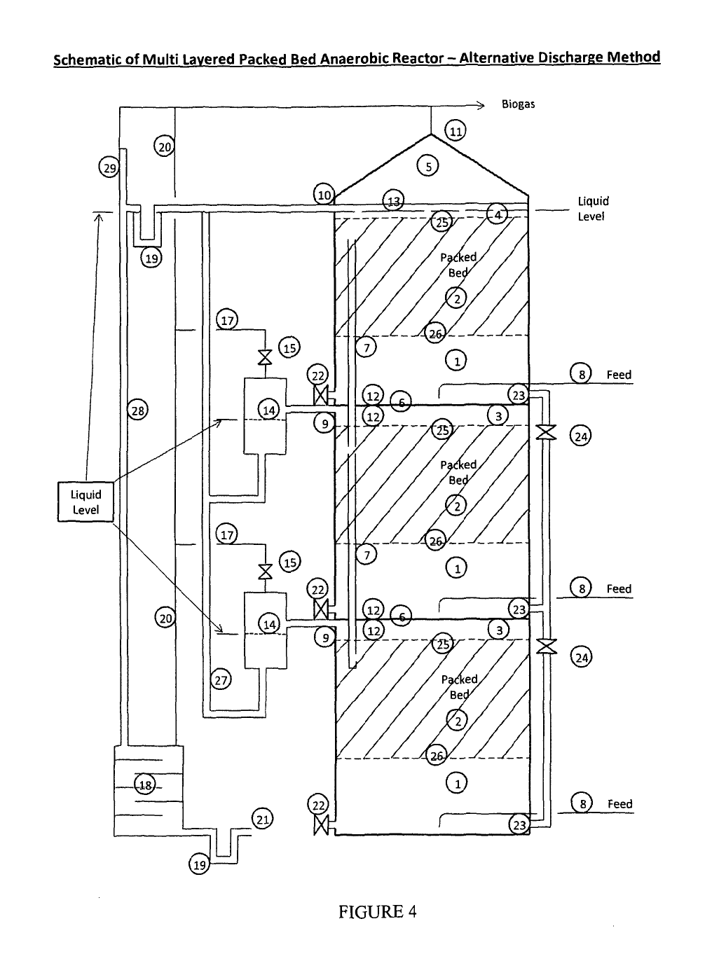

FIG. 4 shows a further embodiment of a packed bed reactor according to the invention. The reactor is a multi-layered packed bed reactor comprising an alternative arrangement for discharge of liquid and gases from the chambers to that illustrated in FIG. 3.

The reactor of the invention comprises two or more reaction chambers arranged one above the other. It is preferred that the chambers are arranged in a substantially vertical (preferably vertical) configuration. In one embodiment, therefore, the reactor of the invention comprises one or more stacks, or one or more substantially vertical stacks, of reaction chambers.

The reaction chambers of the reactor are separated from one another. In particular, there should be no flow of bacteria, biomass or of the products of anaerobic digestion between chambers. Each chamber operates independently to produce biogas by anaerobic digestion of biomass. Each chamber is separated from the next, for example by a solid plate. As outlined above, although the chambers can be considered to be discrete chambers which operate independently, there is generally a pressure equalisation device between each chamber and the chambers above and below it in the reactor. The pressure equalisation device allows a very limited flow of treated liquor from each chamber to an adjacent chamber in order to equalise the pressure above and below the solid member separating the chambers.

In general, it is preferred that each chamber comprises one or more of the following features:
(a) A feed distribution system which is operable to distribute the feed substantially evenly across the base of the chamber and which also directs the flow of feed (that is, organic material being fed into the reactor) towards the separation member or plate, in order to stir up and mix the anaerobic sludge with the incoming feed;
(b) A discharge arrangement to remove excess sludge as necessary;
(c) Means to extract samples of the chamber's contents if required;
(d) One or more gas sparge devices to allow the use of biogas or other inert gases to improve mixing or for any other suitable purpose;
(e) One or more pressure sensing devices, preferably situated generally at the top and bottom of the chamber;
(f) Pressure equalisation devices providing limited interconnection with the chambers immediately adjacent;
(g) One or more pressure relief valves;
(h) Temperature sensor;
(i) pH sensor;
(j) One or more gas and liquid discharge points (or take-offs);
(k) One or more access inspection points; and
(l) One or more fail-safe discharge points.

It is particularly preferred that each chamber except the uppermost and lowermost chambers have all of the above features. In the packed bed embodiment of the digester, it is also highly preferred that each chamber comprises one or more packed bed containing devices, or grids, in order to maintain the correct position of the packing material.

The lowermost chamber is generally as described above, but with a single pressure sensing device, preferably situated generally towards the top of the chamber.

The uppermost chamber will also be as generally as described above. In addition, the uppermost chamber preferably comprises one or more means to disperse any foam that may arise during processing, for example one or more spray bars. In addition, the uppermost chamber preferably has only one pressure sensing device, situated generally towards the bottom or base of the chamber. Furthermore, the uppermost chamber preferably has separate liquid take off and gas take-off or discharge points, rather than a combined gas/liquid discharge device, and a conical roof. The liquid, discharge point is preferably situated at the maximum liquid level. The gas discharge point is preferably situated at the top of the reactor, i.e. at the point of the cone. Nonetheless, in embodiments in which the reactor comprises three or more reaction chambers, the uppermost chamber may comprise a combined gas and liquid discharge point and a flat roof.

The number of reaction chambers, or layers, present in the reactor may be varied to suit individual requirements and processing conditions. Any suitable number of reaction chambers may be present. However, the reactor preferably comprises two or more reaction chambers, more preferably three to five reaction chambers arranged one above the other. As the number of reaction chambers arranged one above another increases, the yield of biogas per unit area of the reactor footprint increases. The optimum number of chambers depends on the characteristics of the feed stock, or organic material, being applied, the processing parameters required and on various engineering and commercial considerations. For example, depending on the site of the reactor, ground loading factors may need to be considered, for example whether there is any need to provide piled foundations for a reactor comprising more than a certain number of chambers. Engineering fabrication and cost limitations may also need to be considered. Other relevant factors will be apparent to the skilled person.

As illustrated schematically in FIG. 3 for the packed bed embodiment of the invention, each of the stacked reaction chambers preferably comprises a mixing space (1) containing, or suitable for containing, a feed line or feed distribution system (8). The feed distribution system (8) is operable to deliver and distribute feed to the base of the chamber and to mix it with the anaerobic bacteria. Typically, the feed distribution system (8) comprises a series of pipes comprising holes or nozzles through which the feed is delivered. Whilst the Figure shows only one discharge point for each chamber, the number of discharge points may be varied and should be selected according to the area of the base of the chamber. The mixing space (1) enables mixing of the feed with the digester contents, including the anaerobic bacteria. Above this mixing space (1), there is typically provided a packed bed (2) with an upper packing grid (25) and a lower packing grid (26). The packed bed (2) contains packing media and anaerobic bacteria.

The feed within the mixing space (1) is converted to predominantly volatile fatty acids, hydrogen and carbon dioxide by the anaerobic bacteria contained in the chamber. The higher chain volatile fatty acids are further converted to acetic acid by acetogenic bacteria. The acetic acid, hydrogen and carbon dioxide fomed are known as partially processed feed. The partially processed feed passes through the packed bed (2) and is converted to biogas by acetoclastic and hydrogen-utilising methanogens contained in the packed bed (2). (It is possible that some methanogens will remain in the mixing space (1) and also that other bacteria in addition to the methanogens will populate the packed bed (2).)

Any suitable packing media can be used in the packed bed (2). It is preferred that the packing medium has a high surface to volume ratio, in order to provide a suitable environment for methanogenic bacteria. Advantageously, the packing medium has a surface area to volume ratio of above 100 m² per m³, more preferably 200 m² per m³ or above. A suitable packing medium is, for example, Cascade Filterpack™ which is commercially available from Veolia Mass Transfer. The packed bed (2) can be of any suitable depth, however a packed bed depth of about 2 to 3 metres is particularly preferred.

Above the packed bed (2) there is preferably a further space (3) for receiving the processed liquor and biogas produced by anaerobic digestion taking place in the packed bed. Each chamber is further provided with at least one take off (9, 10, 11) for the processed liquor and biogas, typically at the very top of the chamber. By “take off for processed liquor and biogas”, we mean an outlet, discharge, or other means for removing biogas and processed liquor from the chamber.

The uppermost chamber of the bioreactor preferably also comprises a space (1) containing, or for containing, a feed distribution system. Above this space (1), there is preferably provided a packed bed (2), in the same arrangement as the lower chambers described above. The uppermost chamber preferably further comprises separate spaces for liquid and gas (4, 5) and take offs (10, 11) for processed liquor and biogas, respectively. The two take-offs conveniently allow the processed liquor and biogas produced in the uppermost chamber to be taken off separately. The processed feed in the uppermost chamber passes to a liquid transit space (4) above the packing (2). The gas separates from the liquid and passes to the gas space (5), which gas space (5) may be located within a conical roof on the uppermost chamber. The gas exits via the biogas outlet (11) to the further gas line (20). The liquor passes through a perforated pipe (13) into the treated liquor collection pipe (10) and is discharged through a water seal (19) to the de-gassing vessel (18).

It is highly preferred that the pressure at the top of each chamber is equal to the pressure at the bottom of the chamber immediately above it. Therefore, each chamber preferably comprises pressure sensors (12), suitably positioned above and below the plates (6) separating each chamber, so that the pressures immediately above and below each plate can be kept equal so as to avoid collapse of the plate. The bioreactor preferably further comprises pressure equalisation devices (7) between each chamber, to ensure that pressure at the top of one chamber is equal to the pressure at the bottom of the chamber immediately above it. Any suitable pressure equalisation device may be used. Typically, the pressure equalisation device comprises a small bore tube passing through the separation member or plate (6), providing limited interconnection between the chambers. Advantageously, the ends of the tube are positioned within the adjacent chambers such that unprocessed liquor cannot pass between chambers, and also to prevent gas passing between chambers. In particular, the lower end of the tube (that is, the end in the lower chamber) is suitably positioned at a sufficient distance below the separation plate (6) to prevent any gas passing from the lower chamber to the chamber above, and additionally positioned high enough to only pass processed liquor, and no unprocessed liquor, to the chamber above. The upper end of the tube (that is, the end in the chamber above) is suitably positioned sufficiently close to the top of the chamber above so as to pass processed liquor downwards yet sufficiently far from the top of that chamber to ensure that the tube is always flooded.

Suitably, each chamber comprises a removal point (22) for any surplus sludge that accumulates during operation of the reactor. Suitably, the removal point (22) is located at or near the base of each chamber.

The reactor preferably comprises a failsafe device comprising emergency liquor exit/entry points (23) and actuated valves (24). In the event of a failure of the control system or in the event of a power failure, the valves “fail” to the open position, thereby enabling the pressures between the chambers to be properly maintained.

In use, the reactor preferably operates as follows.

Input biomass, or feedstock, is provided. Any suitable input biomass, or feedstock, may be used. In particular, the reactor of the invention may be used to convert the organic material in waste water into biogas. Alternatively, the reactor may be used to convert a specially made solution of organic material to biogas.

Usually, the feed, or input biomass, enters the bottom of each chamber, typically via a feed distribution system within the chamber. The feed is typically mixed with the digester contents in the mixing space (1) at the base of the chamber and is converted to partially processed feed. The partially processed feed passes through the packed bed (2) and is converted into processed feed and biogas, which are collected in the space at the top of each chamber (3) and then discharged from the reactor via a pressure regulating device or pipe (9) to a phase separation vessel (14).

In the phase separation vessel (14), the gaseous and liquid phases may be separated and separately discharged, as shown in FIG. 3, through actuated pressure sustaining valves (15, 16). The actuated valves (15, 16) are advantageously controlled to maintain equal pressure above and below the solid member (6) separating the chambers. The phase separation vessel (14) is preferably fitted with spray bars (not shown) to reduce any foaming. The gas passes via a gas line (17) to a further gas line (20). The liquid phase passes to a de-gassing vessel (18) where any remaining gas is removed, for example by cascading the liquor over a series of plates. The degassed liquor then exits (21) via a water seal (19). Gas from the de-gassing vessel is discharged via further gas line (20).

Alternatively, the gaseous and liquid phases may be discharged as shown in FIG. 4. In this embodiment, the pressure of the liquid phase is sustained by passing the liquid phase from the phase separation vessel to a pipe (27) that extends vertically or substantially vertically to the level of the maximum liquid level of the uppermost chamber. Optionally, pipe (27) intersects or combines with the liquid discharge (10) from the uppermost chamber. The pipe (27) suitably then passes through water seal (19) and is connected via pipe (28) to the de-gassing vessel (18). Preferably, pipe (28) and/or pipe (27) is provided with a siphon break (29).

Further suitable arrangements for discharge of the liquid and gaseous phases will be apparent to the skilled person.

The uppermost chamber preferably operates in the same way as the other chamber(s) with the exception that on passing to the top of the chamber, above the packed bed, the processed liquor and gas are typically discharged separately (10, 11). Since the processed liquor and gas are typically discharged separately from the uppermost chamber, the processed liquor and gas produced in the uppermost chamber does not normally pass through a phase separation vessel (14). However, in embodiments in which the uppermost chamber comprises a combined gas and liquid discharge point, the processed liquor and gas produced in the uppermost chamber will pass through a phase separation vessel (14).

The embodiments described above in connection with FIGS. 3 and 4, are equally applicable to fluidised bed reactors and hybrid reactors as well as packed bed reactors.

The biogas produced by a method employing the reactor of the invention, or by the method of the invention, may be used, for example, in a boiler or in a combined heat and power (CHP) system to generate renewable energy. Surplus heat from the CHP system or some of the heat from the boiler may be used to maintain the optimum process temperature in the reactor chambers. Biogas may also be used in a number of other applications, including as a fuel for gas turbines to generate power; in compressed gas or liquid form as a vehicle fuel; for cooking; and (after purification of the methane) to supplement or blend with natural gas supplies.

1. An anaerobic reactor comprising two or more discrete reaction chambers arranged one above the other.

2. An anaerobic reactor according to claim 1, wherein the reactor comprises an outer housing.

3. An anaerobic reactor according to claim 2, wherein solid members located within the outer housing separate adjacent chambers from one another.

4. An anaerobic reactor according to claim 1, further comprising pressure regulating devices positioned at the discharge from each chamber.

5. An anaerobic reactor according to claim 1, further comprising pressure equalisation devices located between adjacent chambers.

6. An anaerobic reactor according to claim 3, wherein the reactor is in the form of a tank, said tank being separated into discrete chambers by means of the solid members.

7. An anaerobic reactor according to claim 1, wherein the reactor is in the form of separate and stackable chambers.

8. An anaerobic reactor according to claim 1, wherein the reactor is a packed bed reactor, optionally a multibed reactor.

9. An anaerobic reactor according to claim 8, wherein each chamber comprises a feed distribution system, a packed bed, and means for biogas and liquid discharge.

10. An anaerobic reactor according to claim 1, wherein the reactor is a fluidised bed reactor.

11. An anaerobic reactor according to claim 10, wherein each chamber comprises a feed distribution system, a fluidised bed, and means for biogas and liquid discharge.

12. An anaerobic reactor according to claim 1, wherein the reactor is a hybrid reactor comprising at least one packed bed reaction chamber and at least one fluidised bed reaction chamber.

13. A hybrid reactor according to claim 12, wherein the reactor further comprises means to assess input biomass prior to processing and means to divert the input biomass to either a packed bed reaction chamber or a fluidised bed reaction chamber depending on the nature of the input biomass.

14. An anaerobic reactor according to claim 1, further comprising a feed distribution system, wherein, in use, the feed distribution system distributes feed substantially evenly across the base of each chamber.

15. An anaerobic reactor according to claim 1, further comprising a discharge arrangement to remove excess sludge formed within one or more of the chambers.

16. An anaerobic reactor according to claim 1, further comprising means to extract samples of the contents of one or more of the chambers.

17. An anaerobic reactor according to claim 1, further comprising one or more gas sparge devices.

18. An anaerobic reactor according to claim 1, further comprising one or more pressure sensing devices, wherein each device is preferably located substantially at the top or substantially at the bottom of one of the chambers.

19. An anaerobic reactor according to claim 1, further comprising one or more pressure relief valves.

20. An anaerobic reactor according to claim 1, further comprising one or more temperature sensors.

21. An anaerobic reactor according to claim 1, further comprising one or more pH sensors.

22. An anaerobic reactor according to claim 1, further comprising one or more access inspection points.

23. An anaerobic reactor according to claim 1, further comprising one or more fail-safe discharge points.

24. An anaerobic reactor according to claim 1, wherein at least the uppermost chamber comprises separate means for biogas discharge and liquid discharge.

25. An anaerobic reactor according to claim 24, further comprising means to disperse foam in the uppermost chamber.

26. An anaerobic reactor according to any preceding claim 1, wherein at least one of the chambers comprises a combined means for biogas and liquid discharge, and wherein the combined means passes biogas and liquid to a phase separation vessel.

27. An anaerobic reactor according to claim 26, wherein the phase separation vessel comprises means to disperse foam.

28. An anaerobic reactor according to claim 26, wherein the phase separation vessel comprises a pressure sustaining device.

29. A method of producing biogas comprising, digesting biomass in the reactor of claim 1, under anaerobic conditions.

30. The method of producing biogas according to claim 29, wherein the yield of biogas produced per unit area of land increases where occupied by the anaerobic reactor.

31. A method of producing biogas comprising:
a) providing an anaerobic reactor according to claim 1;
b) providing input biomass;
c) carrying out anaerobic digestion of the biomass in the reactor; and
d) collecting the biogas produced.

32. A method of producing biogas according to claim 31, wherein the anaerobic reactor is a packed bed reactor, and wherein the input biomass comprises a liquid with a suspended solids content of from 500 to 1000 mg/l.

33. A method of producing biogas according to claim 31, wherein the anaerobic reactor is a fluidised bed reactor, and wherein the input biomass comprises a liquid with a suspended solids content of greater than 1000 mg/l.

34. A method of producing biogas according to claim 31, wherein the anaerobic reactor is a hybrid reactor, and wherein the input biomass comprising a liquid with a suspended solids content of from 500 to 1,000 mg/l is processed by a packed bed reaction chamber and the input biomass comprising a liquid with a suspended solids content greater than 1,000 mg/1 is processed by a fluidised bed reaction chamber.

35. (canceled)

36. (canceled)

37. (canceled)
